SEQUENCE LISTING (10 sequences)

SEQ ID NO: 1 - DNA, 47 bp, Artificial Sequence
  A primer.
gcggccgcctcgagccatgggggcaggtgccaccggccgcgcgatgg

SEQ ID NO: 2 - DNA, 39 bp, Artificial Sequence
  A primer.
cgcggatccacctcctccgctgttccacctcttgaaggc

SEQ ID NO: 3 - DNA, 45 bp, Artificial Sequence
  A primer.
cgcggatccggtggcggcggaagctcgaataacgctttacaaaca

SEQ ID NO: 4 - DNA, 38 bp, Artificial Sequence
  A primer.
gcggccgcctcgagtgatcaacgtttgtaatcgatggc

SEQ ID NO: 5 - DNA, 24 bp, Artificial Sequence
  A primer.
tagctaatggtgacagggggaatg

SEQ ID NO: 6 - DNA, 26 bp, Artificial Sequence
  A primer.
ctactcaccaatatcttcaaaccatc

SEQ ID NO: 7 - protein, 5 aa, Artificial Sequence
  A linker.
GGGGS

SEQ ID NO: 8 - protein, 10 aa, Artificial Sequence
  A linker.
GGGGSGGGGS

SEQ ID NO: 9 - DNA, 42 bp, Artificial Sequence
  A primer.
aaatgatcaggtggcggcggcagcgtgacagggggaatggca

SEQ ID NO: 10 - DNA, 30 bp, Artificial Sequence
  A primer.
aaatgatcactcaccaatatcttcaaacca

(19) United States
(12) Patent Application Publication
(10) Pub. No.: US 2003/0121068 A1
(43) Pub. Date: Jun. 26, 2003

(54) Vector encoding suicide and marker constructs

(76) Inventors: Paul Orchard, Eden Prairie, MN (US); Victor Lewis, Calgary (CA)

Correspondence Address: SCHWEGMAN, LUNDBERG, WOESSNER & KLUTH, P.A., P.O. BOX 2938, MINNEAPOLIS, MN, 55402, US

(21) Appl. No.: 10/288,250

(22) Filed: Nov. 4, 2002

Related U.S. Application Data
(60) Provisional application No. 60/334,795, filed on Nov. 30, 2001.
(60) Provisional application No. 60/369,507, filed on Apr. 3, 2002.

(51) Int. Cl. A01K 67/027; C07H 21/04; A61K 48/00; C12N 9/06; C12P 21/02; C12N 5/06

(52) U.S. Cl. 800/18; 435/69.1; 435/320.1; 435/354; 435/191; 536/23.2; 424/93.21

(57) ABSTRACT

The present invention provides a vector encoding a detectable cell surface marker and a suicide construct, and cells and a non-human mammal transduced with this vector. Introduction of lymphocytes transduced with this vector, after allogeneic bone marrow transplantation, serves to treat or prevent complications from the bone marrow transplant, including graft versus host disease.

### CROSS-REFERENCE TO RELATED APPLICATIONS

This application claims the benefit of the filing dates of U.S. Provisional Applications Serial No. 60/334,795, filed Nov. 30, 2001, and No. 60/369,507, filed Apr. 3, 2002, under 35 U.S.C. §119(e), which are herein incorporated by reference.

### STATEMENT OF GOVERNMENT RIGHTS

This invention was made, at least in part, with a grant from the Government of the United States of America (National Marrow Donor Program (NMDP) and the Health Resources and Services Administration (HRSA)). The Government may have certain rights to the invention.

### FIELD OF THE INVENTION

The present invention provides a vector including a nucleic acid sequence encoding a detectable cell surface marker and a suicide construct, and cells, e.g. lymphocytes, transduced with this vector. Introduction of lymphocytes transduced with this vector, after allogeneic bone marrow transplantation, serves to treat or prevent complications from the bone marrow transplant, including graft versus host disease.

### BACKGROUND OF THE INVENTION

Patients having blood, lymphatic or bone-related disorders may receive a bone marrow transplant (BMT). Bone marrow taken from the patient is “autologous ” marrow, and bone marrow from an identical sibling (twin) is “syngenic” marrow. Unfortunately, in many circumstances, bone marrow from these sources is unavailable or not appropriate. Donor bone marrow must then be taken from a donor other than the patient or an identical sibling. This type of marrow is termed “allogeneic” bone marrow. Allogeneic BMT is used to treat many hematologic malignancies, such as leukemia, lymphoma and multiple myeloma, as well as to treat genetic disorders. It is the only curative therapy for chronic myeloid leukemia (CML). Allogeneic BMT (allo-BMT) is an important modality in the treatment of hematologic malignancies. In cases of relapsed leukemia, the infusion of matched or alternate donor hematopoietic stem cells after high dose conditioning chemotherapy, with or without radiation, may offer the best chance for permanent cure. Major contributors to non-relapse mortality include infections and the presence of graft-versus-host disease (GVHD). The morbidity associated with GVHD is high, and the mortality observed in cases of severe (grade III/IV) GVHD is greater than 50%. The risk of grade III-IV GVHD is higher in alternative donor transplantation.

The use of T-cell depletion (TCD) has been shown to decrease the incidence and severity of GVHD, although TCD has been associated with an increase in the rate of graft failure and relapse due to a decrease in the protective graft versus leukemia (GVL) effect. The potency of GVL effects is evident from the increased risk of relapse when identical twin donors are used in transplantation, and by comparisons of sibling versus unrelated hematopoictic cells transplants. The curative effects of donor lymphocyte infusions (DLI) in cases of relapsed CML after hematopoietic cell transplantation is additional evidence for the important immunologic role of T-cells in eradicating residual leukemia. Therefore, the inclusion of T cells with an allogeneic graft mediates both beneficial (GVL and increased engraftment) and detrimental (GVHD) effects following hematopoietic cell transplantation.

The therapeutic promise of delayed introduction of donor T lymphocytes following allo-BMT remains limited by GVHD. The predominant therapy used to treat GVHD is global immune suppression. However, immune suppressive therapy increases the risk of infectious complications. Thus, the threat of GVHD must be weighed heavily against the therapeutic effects of allo-BMT, thereby limiting the applications in which the therapy is employed. Accordingly, a regimen for preventing and for treating GVHD is highly desired in order to permit the beneficial use of delayed introduction of donor T lymphocytes following allo-BMT.

### SUMMARY OF THE INVENTION

The present invention provides a method useful for bone marrow transplant that will enhance engraftment, decrease relapse, and enhance immune reconstitution. It is desired to make allogeneic bone marrow transplantation more efficacious, safer, and available to a larger number of patients. The present invention provides a method of treating or preventing complications associated with delayed introduction of T lymphocytes to a patient having previously received an allo-BMT depleted of T lymphocytes.

The present invention also provides chimeric (fusion) constructs that can be used in vectors for expression in eukaryotic cells. The present invention also provides chimeric (fusion) proteins encoded by the fusion constructs. The term “chimeric” is used to mean a sequence or segment including at least two nucleic acid sequences or segments from species that do not combine under natural conditions, or sequences or segments that are positioned or linked in a manner that does not normally occur in nature.

The fusion constructs contain a first region encoding an extracellular domain that allows identification or selection, and a second region encoding a cytosine deaminase (CD) capable of conferring a negative selectable phenotype on cells transduced with the vector, operably linked to the first region. The identification or selection can be by, for example, fluorescence activated cell sorting or magnetic sorting (immunobeads). Examples of suitable extracellular domains include human or murine extracellular domains, such as human proteins, including the nerve growth factor receptor (NGFR), the p75 subunit of NGFR, or CD34, and murine proteins such as Thy1. The CD can be from a prokaryotic or eukaryotic source. In some embodiments, the nucleic acid encoding the CD can be from a yeast, such as Saccharomyces. For example, the CD can be from Saccharomyces cerevisiae. The CD can also be humanized CD. See, e.g., S C Makrides, Protein Expression and Purification 17:183-202 (1999).

The fusion constructs can further contain a third region that increases effectiveness of the CD. For example, introduction and expression from this third region may cause cells to become more susceptible to 5-fluorocytosine (5-FC). The third region may encode a uracil phosphoribosyltransferase (UPRT). In some embodiments of the invention, the UPRT is a Toxoplasma gondi UPRT (Donald et al., PNAS USA, 92, 5749-5753 (1995)). In some embodiments of the invention, the UPRT is a Saccharomyces cerevisiae UPRT (Erbs et al., Cancer Research, 60, 3813-3822 (2000)). In some embodiments, the region encoding the UPRT is included in a single fusion construct that also includes regions encoding CD and NGFR. In some embodiments, a single vector independently includes the NGFR/CD construct and the UPRT construct. In some embodiments, the regions encoding the UPRT and the CD/NGFR are introduced into cells from separate constructs.

Additionally, or alternatively, the vector may contain another nucleic acid sequence region operably linked to another region, which imparts a therapeutic phenotype.

The present invention also provides an isolated, implantable cell containing a vector described above. The cell may be a T-lymphocyte.

The present invention further provides a transgenic non-human mammal containing a vector, cell, nucleic acid sequences, or protein described above. For example, the mammal may be a mouse.

The present invention provides a method of eradicating genetically engineered cells transplanted into a patient including administering 5-FC to the patient.

Moreover, the present invention provides a cell sensitive to 5-FC that includes a chimeric nucleic acid vector that includes a first region encoding a transmembrane and extra-cellular domain of human NGFR, and a second region encoding a Saccharomyces cerevisiae cytosine deaminase (CDs) that confers a negative selectable phenotype on cells transduced with the vector, operably linked to the first region. The cell can be killed using a concentration of 5-FC that can be achieved in vivo in human serum.

The present invention provides a safe, efficient vector for transducing lymphocytes for delayed introduction to a patient. The vector contains at least (i) a construct encoding a selectable cell surface marker and (ii) a suicide construct, which can be utilized in vivo to trigger cell death should a complication correlated with the transduced cells occur.

Using the cells engineered to express the protein of the present invention, it is possible to monitor whether transduced donor lymphocytes introduced post-BMT correlate causally to a complication(s) that arises after their introduction. According to this method, a biological sample is taken from the patient and tested to determine the presence of the marker. The results are correlated against the clinical symptoms of the complication. If a positive correlation is made, then the complication can be treated by specific elimination of the transduced cells. This elimination is achieved by administering a drug that would be modified by protein encoded by the suicide construct, a negative selectable construct whose expression product renders the transduced cell susceptible (directly or indirectly) to cell death. If a negative correlation is made, then it would not be necessary to eliminate the transduced cells.

The present invention also provides a method to treat graft versus host disease that may develop with introduction of transduced lymphocytes into an allo-BMT patient. According to this method, the transduced lymphocytes are made sensitive to a particular agent or drug as a result of expression from the negative selectable suicide construct. Therefore, by administering the appropriate agent/drug to the patient, virtually all of the transduced cells are killed.

In the present invention, a marker is provided for transduction of mammalian cells. In particular, a marker according to the invention can be used in connection with an exogenous construct for insertion into cells, as in the case of gene therapy, in order to monitor the presence of the exogenous construct. In one embodiment, the marker and exogenous construct are in the same reading frame.

Further still, a marker and suicide vector system is provided for transduction of mammalian cells, including human lymphocytes. In particular, a vector containing both a marker and suicide construct according to the invention can be used in connection with a vector system or direct method for insertion into cells, as in the case of gene therapy. When employed together with a means of gene transfer and expression, the marker and suicide construct system permits a clinician or investigator to eliminate the expression of the cells expressing the “therapy” construct in vivo, if desired. In one embodiment, a vector is provided that carries the marker, exogenous “therapy” construct and suicide construct in the same reading frame.

It should be noted that the indefinite articles “a” and “an” and the definite article “the” are used in the present application, as is common in patent applications, to mean one or more unless the context clearly dictates otherwise.

As used herein, the term “protein” includes variants or biologically active fragments of the target protein, such as NGFR, UPRT, or CD. A “variant” of the protein is a protein that is not completely identical to a native protein. A variant protein can be obtained by altering the amino acid sequence by insertion, deletion or substitution of one or more amino acid. The amino acid sequence of the protein is modified, for example by substitution, to create a polypeptide having substantially the same or improved qualities as compared to the native polypeptide. The substitution may be a conserved substitution. A “conserved substitution” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall peptide retains its spacial conformation but has altered biological activity. For example, common conserved changes might be Asp to Glu, Asn or Gln; His to Lys, Arg or Phe; Asn to Gln, Asp or Glu and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cysteine, tyrosine, asparagine and glutamine having uncharged polar side chains; aspartate and glutamate having acidic side chains; and lysine, arginine, and histidine having basic side chains (Stryer, L. Biochemistry (2d edition) W. H. Freeman and Co. San Francisco (1981), p. 14-15; Lehninger, A. Biochemistry (2d ed., 1975), p. 73-75).

It is known that variant polypeptides can be obtained based on substituting certain amino acids for other amino acids in the polypeptide structure in order to modify or improve biological activity. For example, through substitution of alternative amino acids, small conformational changes may be conferred upon a polypeptide that result in increased bioactivity.

One can use the hydropathic index of amino acids in conferring interactive biological function on a polypeptide, wherein it is found that certain amino acids may be substituted for other amino acids having similar hydropathic indices and still retain a similar biological activity.

The variant NGFR protein includes at least 40 amino acid residues, about 100 to about 300 residues, about 200 to about 300 residues, about 265 to about 270 residues, or about 268 amino acids, wherein the variant NGFR protein has at least 50%, preferably at least about 80%, and more preferably at least about 90% but less than 100%, contiguous amino acid sequence homology or identity to the amino acid sequence of a corresponding native NGFR protein.

The variant CD protein includes at least 100 amino acid residues, about 120 to about 200 residues, about 150 to about 160 residues, or about 158 residues, wherein the variant CD protein has at least 50%, preferably at least about 80%, and more preferably at least about 90% but less than 100%, contiguous amino acid sequence homology or identity to the amino acid sequence of a corresponding native CD protein.

The amino acid sequence of the variant NGFR, UPRT, or CD protein corresponds essentially to the native protein amino acid sequence. The NGFR used in the present invention is truncated to remove the intracellular (functional) domain of the encoded protein. As used herein “correspond essentially to” refers to a polypeptide sequence that will elicit a biological response substantially the same as the response generated by native NGFR or CD protein. Such a response may be at least 60% of the level generated by native NGFR, UPRT, or CD protein, and may even be at least 80% of the level generated by native NGFR, UPRT, or CD protein.

A variant of the invention may include amino acid residues not present in the corresponding native NGFR, UPRT, or CD protein, or may include deletions relative to the corresponding native NGFR, UPRT, or CD protein. A variant may also be a truncated “fragment” as compared to the corresponding native NGFR, UPRT, or CD protein, i.e., only a portion of a full-length protein. NGFR, UPRT, or CD protein variants also include peptides having at least one D-amino acid.

The NGFR, UPRT, or CD protein of the present invention may be expressed from an isolated nucleic acid (DNA or RNA) sequence encoding the NGFR, UPRT, or CD protein. Amino acid changes from the native to the variant NGFR, UPRT, or CD protein may be achieved by changing the codons of the corresponding nucleic acid sequence. “Recombinant” is defined as a peptide or nucleic acid produced by the processes of genetic engineering. It should be noted that it is well-known in the art that, due to the redundancy in the genetic code, individual nucleotides can be readily exchanged in a codon, and still result in an identical amino acid sequence.

The terms “protein,” “peptide” and “polypeptide” are used interchangeably herein.

The NGFR, UPRT, or CD protein, as described above, are operably linked to each other. An amino acid or nucleic acid is “operably linked” to another amino acid or nucleic acid when it is placed into a functional relationship with another amino acid or nucleic acid sequence. For example, the NGFR can be operably linked to the CD DNA to generate a single chimeric fusion protein. A promoter or enhancer is operably linked to a coding sequence if it affects the transcription of the sequence. In some embodiments, “operably linked” means that the sequences being linked are contiguous and, in the case of nucleic acid coding sequences, are in reading phase. However, some sequences, e.g. enhancers, do not have to be contiguous to be operably linked. Linking can be accomplished by ligation at convenient restriction sites. If such sites do not exist, synthetic oligonucleotide adaptors or linkers are used in accordance with conventional practice. In some embodiments, a (Gly₄Ser) (SEQ ID NO:7) linker is be used. In some embodiments, this pattern is repeated two or more times into a longer linker.

The vector may be, for example, an adenoviral vector, an adeno-associated virus (AAV) vector, vaccinia virus, moloney-based virus, herpesvirus, murine leukemia virus, a retrovirus, or a lentivirus vector based on human immunodeficiency virus or feline immunodeficiency virus. See, e.g., S C Makrides, Protein Expression and Purification 17:183-202 (1999). The AAV and lentiviruses could confer lasting expression, while the adenovirus would provide transient expression.

### BRIEF DESCRIPTION OF THE FIGURES

FIG. 1 depicts the design of retroviral vectors of the present invention. The NGFR/CDe construct is based on an E. coli CD, and the NGFR/CDs on the Saccharomyces cerevisiae (yeast) CD.

FIGS. 2A and 2B provide data from murine fibroblasts (NIH 3T3; FIG. 2A) and human T cells (CEM; FIG. 2B) transduced with the LNGFR/CDeSN virus and selected in G418 and analyzed for NGFR expression using a biotinylated anti-NGFR antibody and streptavidin PE. The expression of NGFR on wild-type cell lines is so designated; the expression of NGFR on the LNG/CDeSN transduced lines is depicted on the shaded portion of the figure.

FIGS. 3A and 3B provide analysis of NIH 3T3 and CEM cells transduced with the LNGFRSN, LCDeSN and LNG/CDeSN retroviruses which were compared in a colorometric proliferation assay 5 days after the initiation of the culture to determine the number of viable cells present in increasing concentrations of 5-FC. Results are expressed as the percentage of maximal proliferation in conditions devoid of 5-FC. The means of a replicate of 5 wells is depicted as well as the standard deviation at each point. Each assay was repeated at least 3 times, and a representative experiment is presented.

FIGS. 4A and 4B are graphs showing FACS analysis data for NIH 3T3 and human T cells previously transduced with the LNG/CDsSN virus and selected in 0.4 mg/mL G418 by flow cytometry.

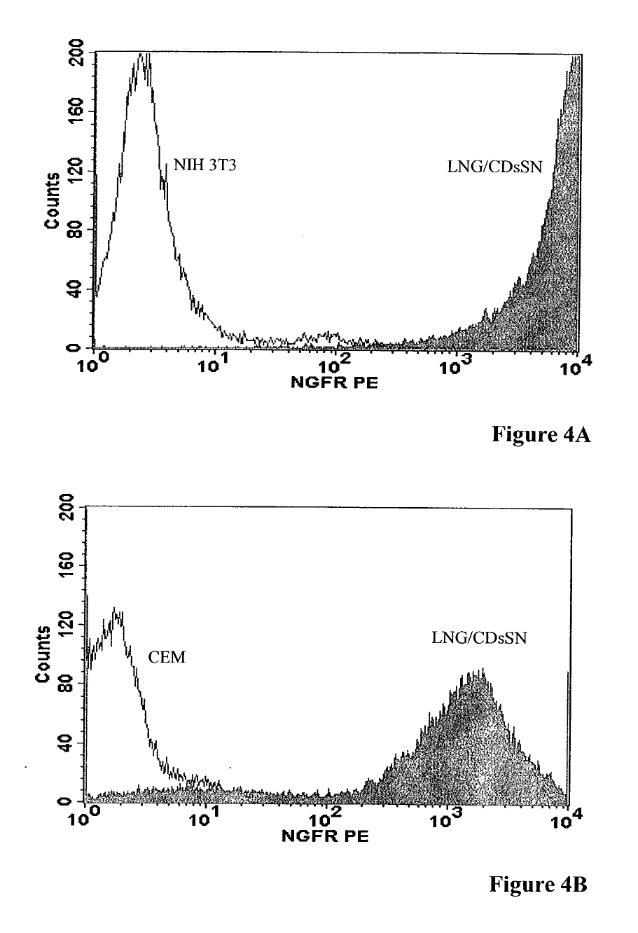

FIG. 5 is a graph showing antigen density in control cells, and cells transduced with LNG/CDeSN or LNG/CDsSN.

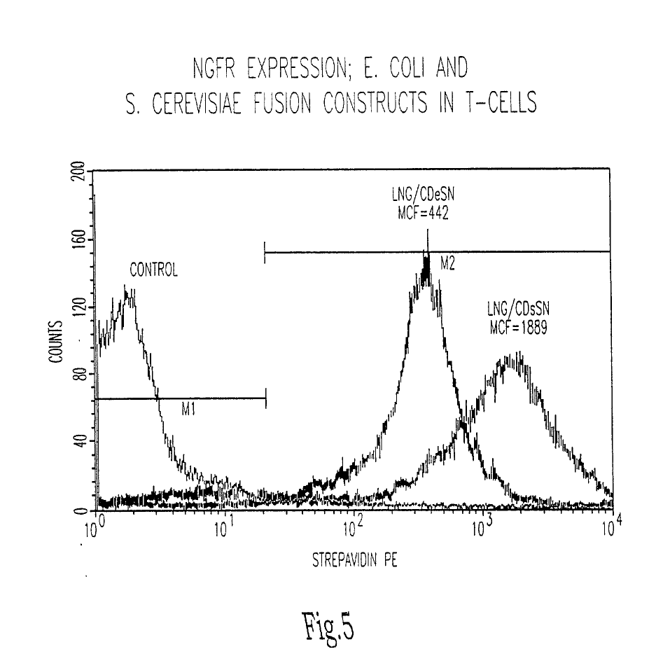

FIGS. 6A and 6B give comparative analysis in NIH 3T3 and CEM cells transduced with the LNG/CDeSN, LCDsSN and LNG/CDsSN retroviruses. Standard deviations of each point are presented in the proliferation assay.

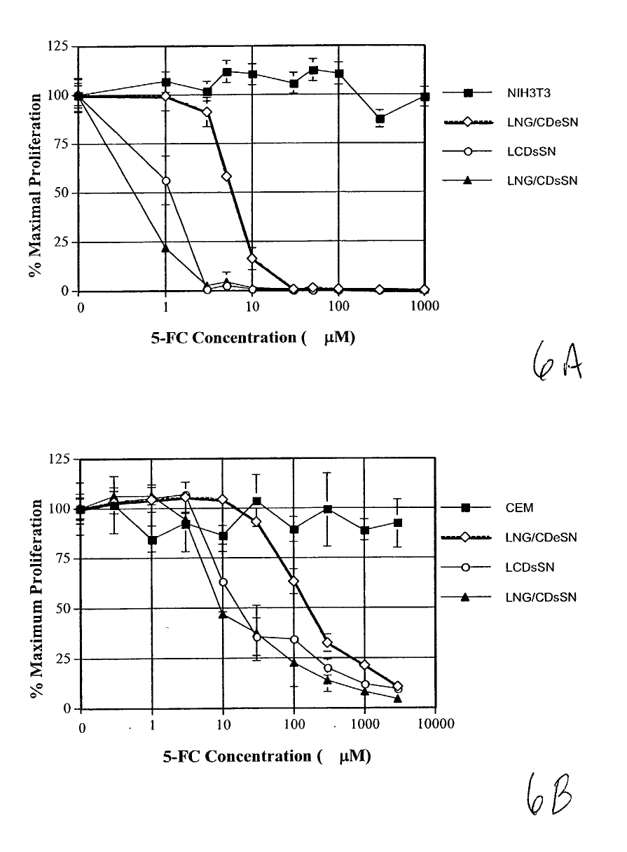

FIG. 7 presents survival statistics for mouse groups injected transduced cells. The saline group represents animals that received injections without the CEM cells.

FIG. 8 presents comparative analysis of the conversion of 5-FC to 5-FU.

FIG. 9 shows the in vivo effects of 5-FC on tumor size in mice.

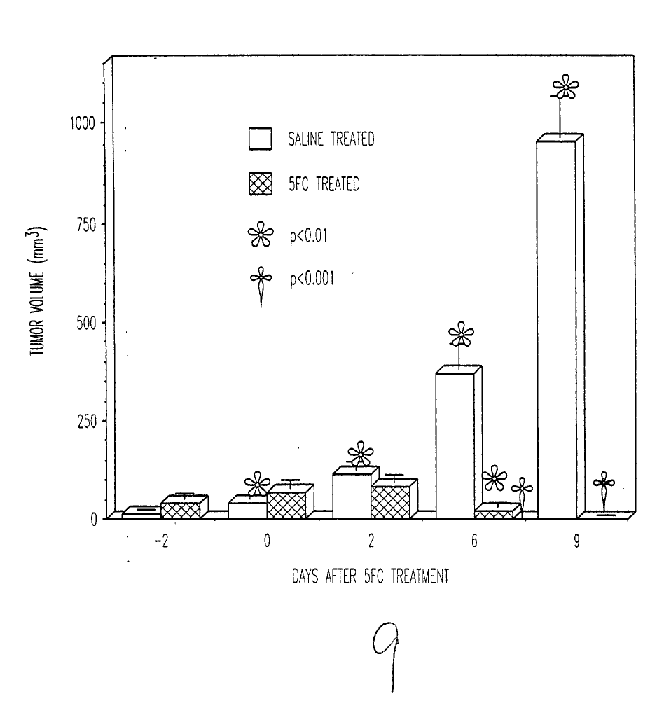

FIG. 10 depicts a comparative analysis of CEM cells transduced with the LNG/CDsSN retrovirus against CEM cells also transduced with a retrovirus containing the URPTase construct.

### DETAILED DESCRIPTION OF THE INVENTION

In delayed lymphocyte introduction therapy, there is a need for a simple method of monitoring the lymphocytes post-infusion. Effective monitoring would permit an investigator to determine whether the infused lymphocytes contribute to or cause a variety of complications that may occur after infusion. Since complications post-BMT can arise from a variety of origins, and since the patients are highly immuno-suppressed, rapid determination of the mechanisms underlying complications is highly desired.

Recently, investigators have transduced lymphocytes for delayed introduction with a selectable marker gene for neomycin phosphotransferase (neo). Thereafter, PCR was employed to monitor the gene in cells-biopsied from the patient. However, this method is cumbersome and PCR is time consuming. A vector expressing a marker construct, to be useful in the present context, should be safe, efficient, and preferably should not substantially interfere with the lymphocyte's range of immune functions or its longevity (persistence) in the recipient's immune/circulatory system.

Hence, it is desired to provide a vector carrying a marker that permits efficient and fast expression, and easy detection of cells carrying the vector by methods such as fluorescence activated cell sorting. Furthermore, easy monitoring after infusion (particularly of peripheral lymphocytes) and nonimmunogenicity would also be desirable. Additionally, it would be beneficial to provide within the same vector a “suicide” construct, which would enable allow for the killing of the cells carrying the vector. This would permit better diagnosis of complications, and therefore, more successful treatment.

T-cells genetically engineered prior to transplantation to facilitate eradication in case of GVHD provide an added measure of safety following allogenic transplantation. As retroviral transduction remains inefficient, a construct allowing positive as well as negative selection is necessary.

Strategies that would permit the inclusion of T-cells within the graft while allowing additional control over GVHD enhance the success of allogeneic BMT. Genetic engineering of T-cells obtained from the donor prior to transplantation to express a “suicide construct” is one way to control GVHD while allowing the presence of these cells in the graft. The herpes simplex virus thymidine kinase (HSV-tk) has been studied extensively for its function as a suicide construct in engineered T-cells and is being tested in current clinical trials. The HSV-tk construct product converts the monophosphate form that can be further phosphorylated to the triphosphate form that competes with thymidine, leading to DNA arrest by preventing subsequent chain elongation.

There are several limitations to the use of HSV-tk in clinical trials. Immunogenicity of engineered T-cells expressing the HSV-tk construct and in patients with AIDS has been demonstrated in allogeneic transplantation. In addition patients with viral infections requiring treatment with ganciclovir (GCV) demonstrated clearance of manipulated T-cells, thus diminishing the beneficial effects of their presence. There has been an additional report of resistance to GCV in a patient with chronic GVHD. This was attributed to the ineffectiveness of the cell cycle dependant HSV-tk in chronic GVHD, which may be caused by very slowly proliferating lymphocytes. HSV-tk has minimal effects on non-proliferating cells, as interfering with DNA synthesis may not impact these quiescent cells. More recently, demonstration of a 227-bp fragment deletion in HSV-tk construct in subcloned cells has contributed to GCV resistance in transduced cells. Based on these observations, it is clear that HSV-tk is not the most suitable candidate for suicide gene engineering and that the use of other genes needs to be explored.

Further, in previous studies, the HSV-tk construct and the NGFR construct exist as separate constructs with the HSV-tk construct being the suicide construct, and NGFR being the marker construct. It was therefore possible that selection on the basis of NGFR might result in cells that were not actively expressing HSV-tk. This is an important disadvantage, as the selected population of cells could potentially not be controlled using the suicide gene strategy. In addition, there are concerns that the HSV-tk construct is less able to control the functional aspects of T cells than other potential suicide constructs, such as cytosine deaminase (CD).

CD is expressed in bacteria and fungi and converts the relatively non-toxic agent 5-FC to the very toxic compound 5-fluorouracil (5-FU), which can affect DNA synthesis by further conversion to 5-fluorodeoxyurine-2 monophosphate and triphosphate through cellular enzyme systems. Also, by conversion to 5-fluorouridine triphosphate, 5-FU can impact mechanisms dependant on RNA synthesis. Transduced cells not actively dividing but possibly contributing to GVHD through the production of cytokines can also be impacted using CD-based strategies.

A limitation to using transduced cells for transplantation relates to their selection following the transduction process. Antibiotic resistance gene mediated selection remains a standard procedure for identifying and enriching transduced cell populations. This procedure is time consuming, and in cases where the suicide construct and the selection marker are expressed from separate promoters, positive selection of transduced cells using antibiotic resistance cannot always ensure expression of the suicide construct.

The present inventors constructed a fusion construct (NGFR/CDe) encoding a truncated human nerve growth factor (NGFR) receptor including only the extracytoplasmic and transmembrane domains of NGFR, and the bacterial E. coli CD construct. They also constructed a similar fusion construct (NGFR/CDs) that incorporated the S. cerevisiae CD. The fusion constructs were engineered to encode the extra-cellular and transmembrane domains of NGFR, linked as part of the same construct to the cytosine deaminase. A flexible linker was incorporated between the NGFR and CD regions. These fusion constructs achieved the function of NGFR as well as CD within a single construct. Expression of these chimeric constructs produced fusion proteins that were identified on the cell surface as well as maintaining CD function. NGFR expression was documented by flow cytometry and magnetic bead technology, and was shown to be comparable to the wild-type constructs.

The inventors documented by flow cytometry that the fusion construct expressed NGFR on the cell surface, and that the fusion construct preserved CD function that was comparable to the function of an unmodified CD construct. While this was an important finding, the cells that were of primary interest, human T-cells, could not be easily eradicated in concentration of 5-FC that can be achieved in human serum. This is a critical drawback of CDe. Therefore, the inventors redesigned the NGFR/CD construct to incorporate the CD construct derived from the yeast Saccharomyces (CDs). This construct had been recently shown to have enhanced activity over the E. coli counterpart. In comparison to the previously engineered NGFR/CD E. coli construct, the NGFR/CD Saccharomyces construct is better expressed based on flow cytometric experiments and has enhanced killing in all cell lines that have been tested. The inventors have also discovered that this fusion construct eradicates tumor cells from animals following administration of 5-FC.

Both fusion CD constructs facilitated conversion of 5-FC to 5-FU, and effective cellular toxicity was documented; however, the NGFR/CDs fusion construct was shown to function more effectively than CDe in expression, as determined by flow cytometry as well as in cytotoxicity assays. The use of single CD fusion constructs providing both positive and negative selection are advantageous in gene therapy applications in which purification of transduced cells is important.

The fusion construct described herein combines the truncated human nerve growth factor receptor and Saccharomyces-derived cytosine deaminase to provide an efficient suicide system. It combines the ease of identification of transduced cells with the effective elimination of these cells in the presence of 5-FC. The fusion of the two constructs ensures that engineered cells selected on the basis of NGFR also express the protein with the capacity for negative selection, as they are the same protein. This should assure that cells expressing the NGFR/CD molecule are sensitive to 5-FC. This allows for elimination of all transduced cells when suitable 5-Fc levels are attained in the serum. CD-engineered donor lymphocytes will be effective in preventing GvHD and will be able to circumvent most of the limitations inherent to the HSV-tk/GCV suicide system.

The terms “cell,” “cell line,” and “host cell” include progeny or potential progeny of these designations. A “transformed cell” or “transduced cell” is a cell into which (or into an ancestor of which) has been introduced a nucleic acid molecule of the invention.

In some embodiments of the invention, the nucleic acid molecule is transferred to the cell via the use of viral vectors. However, other vectors may be used to achieve the same result. Thus, the term “transduction”, as used herein, is not limited to viral transduction. The terms “engineered,” “transfected,” “transformed,” and “transduced” are used interchangeably.

A synthetic construct of the invention may be introduced into a suitable cell line so as to create a transfected cell line capable of producing the protein or polypeptide encoded by the synthetic construct. Vectors, cells, and methods for constructing such cell lines are known in the art. The words “transformants,” “transformed cells,” and “transduced cells” include the primary transformed cells derived from the originally transformed cell without regard to the number of transfers. All progeny may not be precisely identical in DNA content, due to deliberate or inadvertent mutations. Nonetheless, mutant progeny that have the same functionality as screened for in the originally transformed cell are included in the definition of transformants.

The term “vector” is used in reference to nucleic acid molecules into which fragments of nucleic acid may be inserted or cloned and can be used to transfer nucleic acid segment(s) into a cell. Vectors may be derived, for example, from plasmids, bacteriophages, viruses, cosmids, and the like.

The terms “recombinant vector” and “expression vector” as used herein refer to DNA or RNA sequences containing a desired coding sequence and appropriate DNA or RNA sequences necessary for the expression of the operably linked coding sequence in a particular host cell. Prokaryotic expression vectors may include a promoter, a ribosome binding site, an origin of replication for autonomous replication in a host cell and possibly other sequences, e.g. an optional operator sequence, optional restriction enzyme sites.

The terms “nucleic acid molecule,” “gene,” “nucleic acid sequence,” “construct,” and “nucleic acid region,” encoding a protein or proteins refer to a nucleic acid sequence that includes a sequence encoding the protein or proteins. The protein can be encoded by a full-length coding sequence, or by any portion of the coding sequence, as long as the desired activity is retained. The coding region may be present either in a cDNA, genomic DNA or RNA form. When present in a DNA form, the nucleotide sequence may be single-stranded (i.e., the sense strand) or double-stranded. Suitable control elements such as enhancers/promoters, splice junctions, polyadenylation signals, etc. may be placed in close proximity to the coding region of the construct if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. In further embodiments, the coding region may contain a combination of both endogenous and exogenous control elements.

The term “transcription regulatory element” or “transcription regulatory sequence” refers to a genetic element or sequence that controls some aspect of the expression of nucleic acid sequence(s). For example, a promoter is a regulatory element that facilitates the initiation of transcription of an operably linked coding region. Other regulatory elements include, but are not limited to, transcription factor binding sites, splicing signals, polyadenylation signals, termination signals and enhancer elements.

Transcriptional control signals in eukaryotes include “promoter” and “enhancer” elements. Promoters and enhancers consist of short arrays of DNA sequences that interact specifically with cellular proteins involved in transcription. Promoter and enhancer elements have been isolated from a variety of eukaryotic sources including yeast, insect and mammalian cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types. For example, the SV40 early gene enhancer is very active in a wide variety of cell types from many mammalian species and has been widely used for the expression of proteins in mammalian cells. Two other examples of promoter/enhancer elements active in a broad range of mammalian cell types are those from the human elongation factor 1 gene and the long terminal repeats of the Rous sarcoma virus and the human cytomegalovirus. The promoter may be a constitutive promoter, or the promoter may be an inducible promoter. The promoter may also be a strong promoter, or the promoter may be a weak promoter. In some embodiments of the invention, the promoter is an osteoclast specific promoter, a lymphocyte specific promoter, or a T-cell specific promoter.

The term “promoter/enhancer” denotes a segment of DNA containing sequences capable of providing both promoter and enhancer functions (i.e., the functions provided by a promoter element and an enhancer element as described above). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a construct by means of genetic manipulation (i.e., molecular biological techniques) such that transcription of the construct is directed by the linked enhancer/promoter.

The presence of “splicing signals” on an expression vector often results in higher levels of expression of the recombinant transcript in eukaryotic host cells. Splicing signals mediate the removal of introns from the primary RNA transcript and consist of a splice donor and acceptor site. A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamH I/Bcl I restriction fragment and directs both termination and polyadenylation.

Eukaryotic expression vectors may also contain “viral replicons ” or “viral origins of replication.” Viral replicons are viral DNA sequences which allow for the extrachromosomal replication of a vector in a host cell expressing the appropriate replication factors. Vectors containing either the SV40 or polyoma virus origin of replication replicate to high copy number (up to 10⁴ copies/cell) in cells that express the appropriate viral T antigen. In contrast, vectors containing the replicons from bovine papillomavirus or Epstein-Barr virus replicate extrachromosomally at low copy number (about 100 copies/cell).

I. The Vector System

In one embodiment of the invention, a vector is provided that has exceptional properties useful in providing expression in mammalian cells for eradication in vivo. This methodology could be employed in the depletion of an engineered population of lymphocytes, or in a malignant cell population such as brain, liver, ovarian, breast, prostate or renal cancer. The engineering of these cells with NGFR/CD will allow rapid testing to evaluate the presence of residual transduced cells by flow cytometry or immunohistochemistry using the NGFR portion of the NGFR/CD product. In particular, the vector according to the invention provides at least a marker for monitoring the presence of the inserted exogenous construct in vivo or ex vivo.

The vector employed in the present method provides a marker and suicide fusion construct to permit elimination of cells engineered to express the exogenous construct (i) in the event that the engineered cells cause complication(s) that outweigh the benefit of the presence of the engineered cells, or (ii) in the event it is desired to terminate the lives of the engineered cells in vivo. Namely, the vector contains a sequence encoding an easily selectable cell surface marker that is an extracellular domain, and also contains a suicide construct, which can be activated in vivo to trigger cell death should a complication correlated with the transduced cells occur.

The cell surface marker according to the invention is an extracellular domain (e.g., the extracellular domain of NGFR) that can allow easy and rapid identification and selection of engineered cells. One method of identification is flow cytometry, which is quantitative and allows the evaluation of sub-populations of cells, especially if lymphocytes have been genetically modified. In addition, sections of tissues can be stained for the presence of NGFR using immunohistochemistry. This technique has been tested and shown to provide documentation of the presence of NGFR in tumor cells injected into mice. A high level of purity can be achieved using this procedure because of the specificity of the antigen antibody binding. Further, the process is less time intensive compared with antibiotic selection. A cell surface marker according to the invention is one that is not normally expressed on the surface of the mammalian cell to be transduced. To permit differentiation among transduced (marked) and unmarked blood cells, for example, a cell surface receptor can be chosen from a set of receptors that are expressed only in brain or spinal tissue (substantially not expressed on blood cells), such as forms of “trk” receptor, or non-immunogenic fetal receptors that are not normally expressed in fully developed humans. Similarly, to permit differentiation among transduced (marked) and umarked cells of the central nervous system, a cell surface receptor can be chosen from a set of receptors that are expressed only in a type of non-CNS tissue, such as an hepatic-specific receptor (e.g., bile acid receptor proteins, LDL receptor, etc).

A preferred marker in this context is a human cell surface receptor molecule that is modified to eliminate the functional activity of the marker. For example, modifications can be made by truncating the receptor or otherwise mutating the portion of the molecule that performs signal transduction. A resultant modified receptor may no longer transduce a signal, yet it retains its binding activity with respect to a cognate antibody or ligand.

Further, a cell surface receptor chosen according to the invention, when applied for use in humans, is expressed by normal human cells. By design, therefore, the modified cell surface receptor, when expressed in transduced human cells, is non-immunogenic. NGFR is less likely to induce a substantial immunologic response, as the protein is human in origin.

A vector according to the invention carries a first region that encodes an extracellular domain that allows identification or selection. Examples of marker genes (constructs) include nerve growth factor receptor (NGFR), Thy1 and CD34. For instance, it can be an NGFR, and may even be a portion of NGFR (i.e., the trans-membrane and extracellular domains of human NGFR). According to the invention, modified NGFR also is useful in many different types of gene therapies (for example, in treating ADA (adenosine deaminase disorder), CF (cystic fibrosis) and a variety of diseases being treated with gene therapy), with the possible exception of treatments specifically targeted to the CNS or brain, where the normal expression of NGFR may make it problematic to differentiate marked from unmarked cells.

A vector according to the invention carries both marker and a suicide construct, which, upon being transduced into a host cell, expresses a phenotype permitting negative selection (i.e., virtual elimination) of stable transductants. The suicide construct of the present invention is a cytosine deaminase, for example from E. coli or S. cerevisiae . The CD may be a humanized CD.

According to the invention, a vector may be a retrovirus, for example, an adenoviral vector, an adeno-associated virus (AAV) vector, vaccinia virus, moloney-based virus, herpesvirus, murine leukemia virus, a retrovirus, or a lentivirus vector based on human immunodeficiency virus or feline immunodeficiency virus. Alternatively, the vector may be a portion of these viruses. Retroviruses have been shown to be useful in gene therapy and are advantageous in the present context for transducing lymphocytes because they infect primarily only dividing cells.

The chimeric vector of the present invention may in some embodiments carry only two constructs, since concerns for safety and non-immunogenicity may mitigate against inclusion of any additional constructs beyond what is necessary for the vector to accomplish its purpose. In embodiments when the chimeric vector is used in gene therapy to introduce a desired exogenous “therapy” construct for therapeutic purposes, the vector preferably carries only three constructs (marker, suicide, and a desired exogenous “therapy” construct).

In methods of treating allo-BMT according to the invention, the cells transduced by the vector (preferably NGFR/CDs) are donor T-lymphocytes. “Donor” means that the cells are from the original donor of hematopoietic cells used in the allogeneic BMT. Hematopoietic cells also are transduced by a vector according to the invention.

In methods of gene therapy in which a vector according to the invention additionally carries an exogenous construct, a multitude of cell types may be transduced. For example, a desired exogenous construct can be inserted into a NGFR/CDs vector using conventional genetic engineering techniques. Depending on the type of gene therapy to be performed, the cell-type will vary. For example, the NGFR/CDs construct can potentially be expressed in many cell types using gene therapy techniques. Examples of target cells that could be engineered to express the NGFR/CDs construct include lymphocytes and malignant tissues.

It is further contemplated that, in methods of gene therapy according to the invention, other vectors in addition to retroviral vector, such as adenovirus-derived vector, can be manipulated to carry the marker and suicide construct of the invention. For example, an adenoviral vector carrying NGFR and the suicide construct CDs is useful to transduce in vivo cells such as lung, bronchial and epithelial cells with a normal exogenous construct to treat bronchio-epithelial diseases (for example, cystic fibrosis).

The starting material (such as a NGFR gene, a CDs gene, and a UPRT gene) used to make the fusion construct of the present invention may be substantially identical to wild-type genes, or may be variants of the wild-type gene. Further, the polypeptide encoded by the starting material may be substantially identical to that encoded by the wild-type gene, or may be a variant of the wild-type gene. The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may include additions or deletions (i.e., gaps) compared to the reference sequence (which does not include additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See http://www.ncbi.nlm.nih.gov. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.”Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may include additions or deletions (i.e., gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide includes a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide includes a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tm is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267 (1984); Tₘ81.5° C+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tm is reduced by about 1° C. for each 1% of mismatching; thus, Tₘ, hybridization, and/or wash conditions can be adjusted to hybridize to sequences of the desired identity. For example, if sequences with >90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tm). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acid Probes, part I chapter 2 “Overview of principles of hybridization and the strategy of nucleic acid probe assays” Elsevier, N.Y. (1993). Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and awash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

II. Selection and Monitoring of Transductants

According to the invention, the use of a cell surface marker provides important advantages over conventional markers for gene therapy, that typically involve nuclear markers (e.g., the gene neo), identifiable only by nucleic acid detection methods such as PCR. In contrast, a cell surface marker permits faster, easier detection ex vivo of cells expressing marker, for example, fluorescence-activated cell sorting (FACS) analysis (F. Mavilio et al., Blood 83, 1988 (1994)). Cell surface markers (i.e., NGFR), allow rapid in vitro selection of transduced cells by the use of magnetic immunobeads conjugated to antibodies (i.e., anti-NGFR antibody).

A biological sample from which the marker can be detected preferably is a set of peripheral blood lymphocytes, but can include biopsied material from the patient in any tissue of the body where the lymphocytes would be expected, particularly for monitoring GVHD signs (for example, skin or liver).

Importantly, the detection methods enabled by use of the present invention are quicker, and permit faster clinical assessment of the infused cells' performance. Additionally, in the initial transduction of cells to be infused, NGFR expresses much faster (1-2 days) than does neo (about 2 weeks). Lymphocytes kept in culture for prolonged lengths of time tend to change shape and diversify. Thus, the time savings gained from using cell surface receptor are beneficial in many ways.

Clinically, it is important for virtually all of the infused donor lymphocytes to carry the suicide construct to ensure efficacious treatment of any GVHD that may develop. Thus, according to the invention, a single selection can be performed either on a set of transduced lymphocytes to be infused to yield, preferably at least 95%-100% transduced lymphocytes (PBLs).

Monitoring of infused transduced donor lymphocytes can be performed, preferably, by taking a sample of the recipient patient's peripheral blood (in preservative free heparin), and using FACS analysis to determine the frequency of cells expressing the surface marker construct and ex vivo characterization of the transduced cells. Confirmation of the presence of transduced donor cells at low frequency, such as in biopsied biological material, is performed by PCR and or reverse PCR.

III. Reconstituting Immunity, Guidelines for Dosage of Donor Lymphocytes

At the time of allo-BMT, recipient patients are severely immunodepressed. Normally, in a drug-induced immunosuppression, such as in organ transplant recipients, removal of pharmacologic immunosuppression will enable fast reconstitution of the immune system. This in not the case in post allo-BMT recipients. For this reason, recurrent or persistent viral infections such as CMV and EBV may be associated with a poor prognosis.

One means of decreasing the morbidity and mortality of allogeneic transplantation is to perform the depletion of T cells. This decreases the incidence and severity of graft versus host disease; however, the recovery of the new immune system is significantly affected by T cell depletion. Following T cell depletion, the infusion of donor T cells engineered to express the NGFR/CDs construct may assist with immune reconstitution, while providing the capacity to control GVHD should it develop through treatment with 5-FC systemically. In this manner, it may prove possible to decrease post bone marrow transplant complications.

In one embodiment of the present invention, the strategy to reconstitute immunity includes the following general regimen. First, transduced donor lymphocytes are prepared according to Examples 1-3. The route of administration preferably is intravenous, although other routes into the circulatory system are contemplated. The lymphocyte preparation is introduced into a suitable patient at the time of transplant or after a delay following post allo-BMT in variable dosages, depending on the patient's general clinical status (what complications are being treated). However, the following general guidelines apply to most patients, to begin at 10⁵ to 10⁸ cells/Kg per body weight per infusion, according to the recipient's condition.

(A) Prevention of Disease Relapse

To prevent disease relapse, transduced donor lymphocytes can be infused every two weeks, beginning at day 30 after marrow reconstitution (ANC<500) at escalating cell doses, beginning at 10⁵ cells/Kg per body weight per infusion, until a total of 10⁷ cells/Kg is reached, or until relapse or GVHD occurs.

(B) Treatment of Disease Relapse

To treat disease relapse, transduced donor lymphocytes can be infused every two weeks, beginning at day 30 after marrow reconstitution (ANC<500) at escalating cell doses, from about 10⁵ cells per Kg body weight per infusion until reaching a total of 10⁸ cells per Kg body weight per infusion, within about eight weeks time from the beginning of treatment. Infusion of donor lymphocytes should be discontinued if GVHD grade II or higher occurs.

(C) Treatment of Epstein-Barr Virus-Induced B Lymphoproliferative Disorders (EBV-BLPD)

To treat EBV-BPLD, transduced donor lymphocytes can be infused at an initial dose of about 0.5×10⁶ to about 1.5×10⁸ cells per Kg body weight per infusion. Infusion of donor lymphocytes may be repeated weekly until complete remission is achieved or until GVHD grade II or higher occurs.

(D) Use to Enhance Engraftment and Immune Reconstitution Following Transplantation

The use of donor derived T cells engineered with the chimeric suicide construct can be used in association with the removal of the donor T cells from the graft. This replacement of the donor T cells provides for enhanced engraftment and immune function, while allowing the eradication of these cells should GVHD develop.

IV. Treatment of Graft Versus Host Disease

If, in monitoring the patient or the patient's transduced donor lymphocytes after infusion, it is found that they are alloreactive with the recipient patient's own cells, then those lymphocytes can be negatively selected for in vivo (by use of the pro-drug and suicide construct) to relieve the complication. If the patient begins to exhibit symptoms of graft versus host disease concurrent with, or within a few days, weeks or months after infusion of the transduced donor lymphocytes, then steps are taken to determine whether a GVHD complication positively correlates with the transduced lymphocytes. For example, bilirubin levels are detected, and these values are correlated with the timing and presence of transduced lymphocyte in the circulating peripheral blood lymphocytes. Additionally, a skin, gut or liver biopsy may be performed and tissues analyzed immunohistochemically and by PCR for presence of transduced donor lymphocytes in affected liver tissues.

Upon positively correlating the complication or graft versus host disease state with the donor transduced lymphocytes, an investigator may administer a drug (such as 5-FC) to facilitate killing of the transduced cells through the action of the suicide construct.

The following examples are intended to illustrate but not limit the invention.

### EXAMPLES

### Example 1

### Retroviral Plasmid Construction

Retroviruses containing the cytosine deaminase and cytosine deaminase fusion constructs

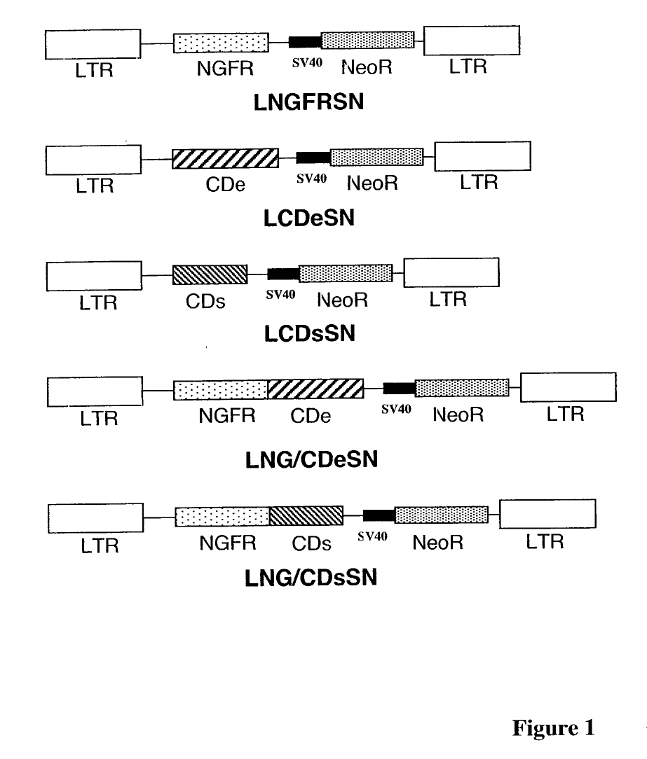

The vectors constructed and tested for this study are depicted in FIG. 1. A plasmid containing the E. coli CD construct was graciously provided by Austin and Huber (Austin and Huber, 1993), was subcloned into a pCR 2.1 vector (Invitrogen Life Technology®, Catalog no. K2000-01) and was sequenced to confirm its identity. The E. coli CD construct (CDe) was used to construct the LCDeSN virus using the LXSN retroviral plasmid (Miller and Rosman, 1989). The truncated human NGFR construct was obtained from the GCsamE75t vector provided by D. Nelson (Orchard et al., 2002). This modification of the NGFR cDNA creates a TAG stop codon at position 250 instead of the cysteine in the wild-type NGFR cDNA, resulting in removal of all but 5 amino acids of the intracytoplasmic region of NGFR. This truncated NGFR construct was amplified utilizing the polymerase chain reaction (PCR) using a sense oligonucleotide (gcggccgcctcgagccATGGGGGCAGGTGCCACCGGCCGCGCGATGG) (SEQ ID NO: 1) designed to introduce NotI, XhoI and NcoI sites for the purposes of cloning. An additional modification was made with this oligonucleotide, converting the C at base 27 to G (underlined), thereby deleting an existing NcoI site in the 5′ portion of the construct while preserving the amino acid sequence (alanine) at this codon. This modification allowed subsequent cloning of the modified construct using the newly created unique NcoI site. An antisense oligonucleotide (cgcggatccacctcctccGCTGTTCCACCTCTTGAAGGC) (SEQ ID NO:2) was designed to delete the TAG stop codon of the truncated NGFR construct while providing additional sequences encoding the first 5 amino acids of a (gly₄ser)₂ (SEQ ID NO:8) linker designed to facilitate three dimensional flexibility between the NGFR and CD domains of the final protein. Incorporated into this oligonucleotide is a BamHI site to allow subsequent ligations. The amplified NGFR construct with these modifications was cloned into the pCR2.1 vector. A sense oligonucleotide with sequences completing the (gly₄ser)2 (SEQ ID NO:8) linker and containing a BamHI site for ligation into the modified NGFR construct was designed, continuing into the E. coli CD construct (cgcggatccggtggcggcggaagcTCGAATAACGCTTTACAAACA) (SEQ ID NO:3) with the exception of the bacterial GTG start codon. An anti-sense oligonucleotide including the stop codon and containing BclI, XhoI and NotI sites (gcggccgcctcgagtgaTCAACGTTTGTAATCGATGGC) (SEQ ID NO:4) was used to amplify the bacterial CD construct. The NGFR and CD constructs were combined into a single fusion construct in pCR2.1 using the BamHI site, and the final fusion construct isolated as a XhoI fragment. This was subcloned into the XhoI site of the LXSN vector and clones screened to confirm the correct orientation. The final construct, termed LNGFR/CDeSN, was transfected into the PA-317 packaging line and G418 (0.4 mg/mL) used to select positive clones (Miller and Buttimore, 1986).

Retroviral vectors incorporating the S. cerevisiae derived CD (CDs) were constructed in a similar fashion. The CDs construct was isolated from S. cerevisiae by PCR using the sense oligonucleotide tagctaatggtgacagggggaATG (SEQ ID NO:5) and the antisense nucleotide CTACTCACCAATATCTTCAAACCATC (SEQ ID NO:6), and subcloned into the PCR 2.1 plasmid. The identity and fidelity of the construct were confirmed by sequencing. The construct was isolated following EcoRI digestion and ligated into the EcoRI site of LXSN to produce the retroviral vector LCDsSN. Construction of the NG/CDs fusion construct was accomplished using the S. cerevisiae derived CD construct in a similar manner to NGFR/CDe. The CDs construct was modified using the sense oligonucleotide (aaatgatcaggtggcggcggcagcGTGACAGGGGGAATGGCA) (SEQ ID NO:9) to introduce a BclI site and the initial portion of the (gly₄ser)₂ (SEQ ID NO:8) polylinker sequence. The antisense primer (aaatgaTCACTCACCAATATCTTCAAACCA) (SEQ ID NO:10) was also designed to contain a BclI cloning site. This modified yeast derived CD construct (CDs) was amplified using PCR and once again cloned into pCR2.1. The bacterial CD sequence was removed from LNGFR/CDeSN using BamHI, and the modified CDs construct isolated from pCR2.1 using Bell and subcloned into the LNG-SN backbone, yielding the LNGFR/CDsSN vector. Viruses containing the wild type truncated NGFR cDNA (LNGFRSN), the wild-type and Saccharomyces CD (LCDsSN) were also constructed to be used as controls.

### Example 2

### Retroviral Transduction

Preparation of Packaging Cell Lines

Retroviral vectors were introduced in the amphotropic PA 317 packaging lines by calcium phosphate transfection. PA 317 cells were placed in 60 mm petri dishes on the first day at a concentration of 5×10⁵. On day 2, a DNA-Ca²⁺ solution (labeled solution 1) was prepared using 5 μg of DNA and 50 μl of 1 M CaCl₂. The final volume of 250 μl was obtained using sterile water. Solution 2 (2×HBS) was prepared using 280 mM NaCl, 50 mM HEPES, 1.5 mM Na₂PO₄ at a pH 7.1. The solution was filtered using 0.22 micron filters prior to transfection experiments and 250 μl of this solution was used for each experiment. Solution 1 was added to solution 2 in a dropwise manner and a precipitate allowed to form over five minutes at room temperature. This precipitate was then placed on the PA 317 cells from day 1 and cells allowed to incubate overnight at 32° C. and 10% CO₂. The cells were exposed to 1 ml of 15% glycerol the next day after which they underwent a thorough washing using PBS. After incubating in 10% CO₂ at 32° C. for two days the cells were split into 100 mm petri dishes at concentrations of 1:4 and 1:10. Stable clonal transfectants were identified using 400 ug/ml of G418. Neomycin resistant clones were tested for NGFR expression by flow cytometry. Transduction of NIH and CEM lines Retroviral supernatants from the PA 317 packaging cell lines were used to transduce fibroblasts (NIH 3T3) as well as human T-cell leukemia (CEM) cell lines. NIH-3T3 cells were maintained in Dulbeccos's Modified Essential Medium (DMEM) supplemented with 10% newborn calf serum (Sigma; St. Louis, Mo.) and penicillin/streptomycin (GIBCO BRL; Rockville, Md.). CEM cells were grown in RPMI supplemented with 10% fetal bovine serum (Sigma; St. Louis, Mo.) and penicillin/streptomycin. Retroviral supernatants were generated from confluent retroviral producing lines in 100 mm petri dishes at 37° C. over 16-24 hours. The supernatants thus collected were spun at 1800 RPM for ten minutes to precipitate any residual producer cells. Transduction of fibroblasts was accomplished by exposing NIH 3T3 parental cells to retroviral supernatants from each of the vectors discussed above in the presence of 8 μg of protamine sulfate/ml. Positive controls were selected using G418 at a final concentration of 400 ug/ml. CEM cell transduction was achieved by exposing 5×10⁵ cells/0.4 ml of media to 0.2 ml of retroviral supernatant in the presence of 8 ug/ml of protamine sulfate. The cells were centrifuged at 3,000×G for one hour and then incubated overnight. New media was added after washing the cells in PBS the next day. The cells were placed in culture for 48 hours, and the percentage of NGFR positive cells was determined using FACS analysis while another aliquot was placed under G418 selection.

### Example 3

### FACS Analysis

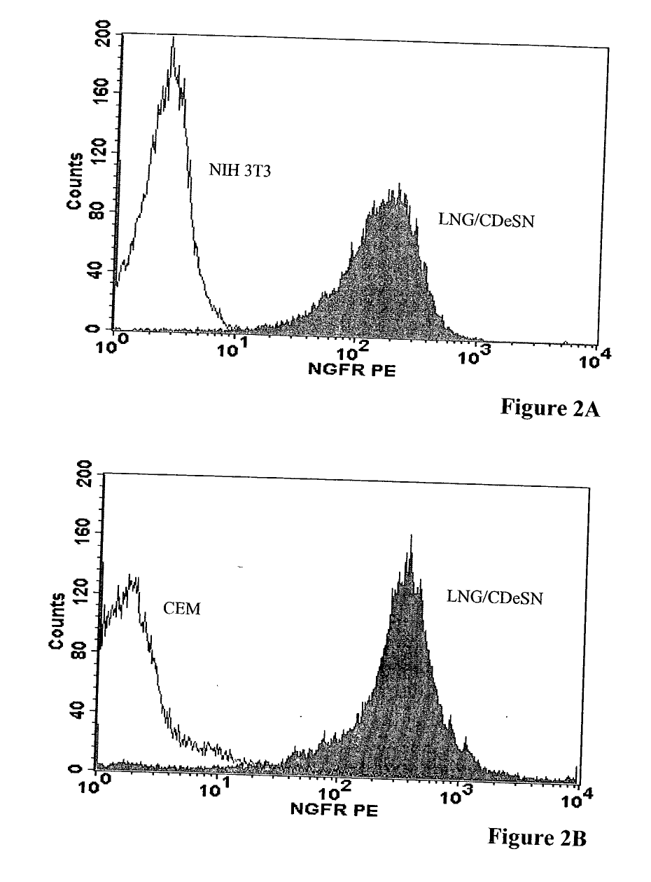

The expression of NGFR from the wild type construct and fusion constructs was determined using a monoclonal antibody to NGFR; the 20.4 hybridoma (murine IgG1) obtained by ATCC (HB 8737, 200-3-G6-4; clone 20.4) was used to prepare the antibody (Taconic BioServices—Germantown, N.Y.). Briefly, the transduced cells (5×10⁵) were exposed to the biotinylated monoclonal antibody for 30 minutes at 4° C. The cells were then washed with PBS twice and counterstained with streptavidin PE (Cat. #349023, Becton Dickinson, Franklin Lakes, N.J.) for 30 minutes at 4° C. FACS analysis was performed using the Becton Dickinson FACSCaliber. FIG. 2 documents the presence of the protein product of the bacterial NGFR/CD construct on the surface of various cell types. The shading represents the genetically engineered cells.

Expression of NGFR, as measured using FACs analytical software, in cell lines containing the various constructs is shown in FIG. 2. In G418 selected population, cells transduced with the NGFR containing vectors but not the ones without show expression of the surface protein. Both NIH 3T3 and CEM cells expressing the fusion construct (LNGFR/CDeSN) were shown to have excellent expression of NGFR as determined by flow cytometry. Cells having identical NGFR expression in the LNGCDeSN lines are clearly present (data not shown) and are selectable but form a smaller proportion within the transduced group of cells. It is clear that the combination of the NGFR with the CD in the fusion protein does not compromise the function of the NGFR component in any way by altering the three dimensional structure of the protein product. This also speaks for the effectiveness of the glycine-serine polylinker used to connect the two constructs in the fusion construct.

### Example 4

### Cytotoxicity Assays

CD function was tested by cytotoxicity assays in which transduced cells are exposed to various concentrations of 5-FC in 96 well plates. Assays were initiated using 500 cells/well of NIH cells or 10,000 cells/well of CEM cells placed in culture with semi-log increasing concentrations of 5-FC for a period of 5 days. An MTS (Promega, Cell Titre 96; cat no. G5430, GI 11) colorimetric assay (based on the conversion of a tetrazolium dye to formazan) was utilized to quantitate the concentration of viable cells on day 5. The absorbance of UV light at a wavelength of 565 nm at a given concentration of 5-FC was used to calculate the LD50 using a μQuant plate reader (Bio Tek Instruments Inc.). The fusion constructs were tested alongside controls containing only the NGFR and the CD constructs.

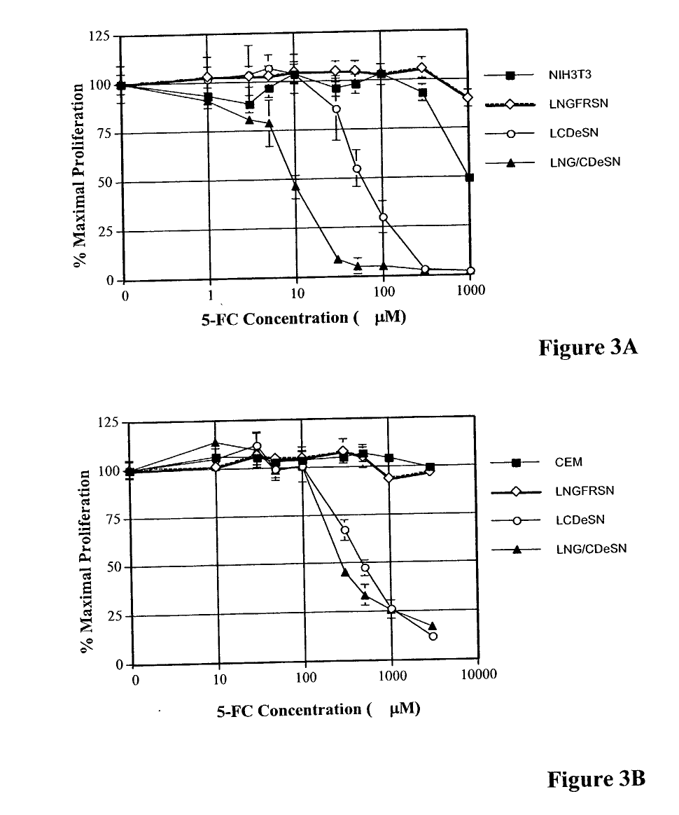

FIG. 3 represents data comparing the wild-type bacterial construct (CDe) with the new fusion construct (NGFR/CDe) in both fibroblasts (3A) and human T cells (3B). The T cell line (CEM) is somewhat more resistant to killing, and this accounts for the different amount of 5-FC used in the two assays.

We tested to determine if the CD construct remained functional in the fusion protein. Cells were incubated with varying concentrations of 5-FC in 96-well plates. After 5 days, MTS reagent was added as per manufacturers recommendations. The reaction involves a color change based on the number of viable cells as the tetrazolium dye changes to formazan. Absorption of UV light at 565 nm can then be used to assess viability (shown in FIG. 3 as percentage of viable cells). Untransduced cells are resistant to the effects of 5-FC. Cells containing the LNGCDeSN or the wild type LCDeSN vector show increased sensitivity that is dose dependant. The fibroblast cell lines expressing the wild type or fusion construct are more sensitive than the human T-cell leukemia lines. Comparisons of the ‘wild type’ and fusion construct construct demonstrates that there is no loss of function when CD is combined with NGFR in the fusion protein.

Thus, the fusion protein retains the function of both components effectively when compared to the wild type constructs. Based on data that yeast derived CD has superior function compared with the bacterial CD (Hamstra et al., 1999), we tested its use in the fusion protein construct. Flow cytometric data on cells transduced with LNGCDsSN demonstrates high NGFR expression in transduced and G418 selection fibroblasts and CEM cell lines.

Cytotoxicity to the cell lines was tested in MTS based cytotoxicity assays described above. Comparisons are made to the wild type LCDsSN virus as well and the bacterial derived LNGCDeSN virus transduced cells. Cytotoxicity with 5-FC in LNGCDsSN transduced cells is clearly superior compared with the bacterial derived fusion protein. The LD50 of 5-FC for LNGCDsSN transduced cells is about one log less compared with the bacterial construct.

### Example 5

### Enzyme Kinetics

Membrane Preparation and Immunoblot

The rate of conversion of 5-FC to 5-FU was tested using HPLC assays. The NIH-3T3 fibroblast cell line was chosen given its large cells size to isolate the enzyme. 2×10⁷ cells were trypsinized and washed in PBS twice. The pelleted cells were swelled in 1 ml of 10 mM Tris-HCl pH 7.5, 1 mM EDTA, 1 tablet of Complete™ protease inhibitors per 10 ml (Roche) on ice for 10 minutes. Cells were lysed by 10 strokes in a glass homogenizer. Nuclear debris was removed by centrifugation at 1,000×g for 10 minutes at 4° C. Membranes were isolated by centrifugation at 75,000×g for 45 minutes at 4° C. Membrane bound proteins were solublized in the above hypotonic lysis buffer with 1% NP-40. Insoluble debris was removed by centrifugation at 16,000×g for 20 minutes at 4° C. Proteins were quantified with the BCA protein assay according to the manufacturers instructions (Pierce, Rockford, Ill.).

Equivalent amounts of proteins were separated by acrylamide gel electrophoresis and transferred to PVDF membrane. NGFR was detected using anti-NGFR from R&D Systems, Inc. (Minneapolis, Minn.) by ECL™ according to the manufacturers protocol (Amersham-Pharrnacia).

Cytosine Deaminase Assays

HPLC assays using tritiated 5-FC were used to assess and compare the rate of conversion of 5-FC to 5-FU in extracts from the membranes of cells engineered to express the NGFR/CDe and NGFR/CDs fusion constructs. A volume of 37.5 uL of membrane extracts from the procedure above (9 mg/mL protein concentration) was incubated at 37° C. with 22.5 μl of cold 5-FC (7000 μM) and 0.5 μl of [6⁻³H] 5-fluorocytsine. Cold 5-FC was used to drive the forward reaction. Reactions were terminated at 0, 1, 4 and 10 minutes using 7 μl of 6M perchloric acid. The precipitate was pelleted by spinning at 15,000 G for five minutes. The reaction was neutralized in using 1M KOH (in 0.5M Tris—pH 7.5). After spinning at 15,000 G for five minutes the supernatant was transferred to fresh tubes for HPLC assays.

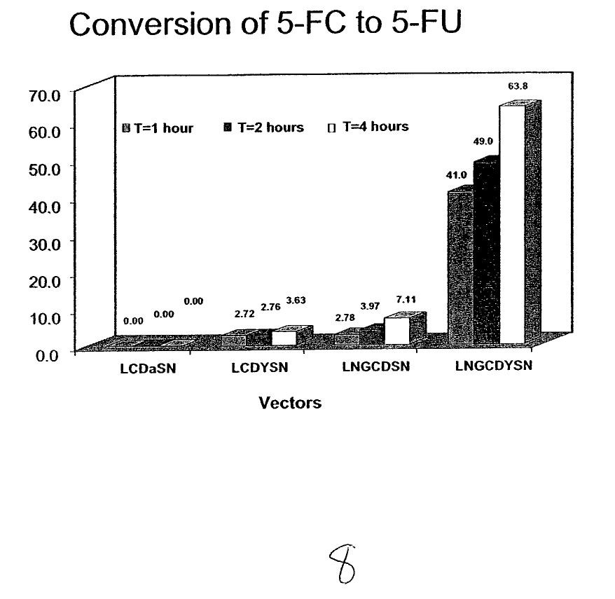

Membrane extracts were tested by HPLC to assess the rate of conversion of labeled 5-FC to 5-FU, and as can be seen in FIG. 8, the enzymatic conversion to 5-FU takes place much more quickly in LNGFR/CDsSN transduced cells than in cells derived from the LCDySN or LNGFR/CDeSN lines. The enhanced conversion of LNGFR/CDySN membrane from 5-FC to 5-FU in comparison to the wild-type CDs construct may be due to the concentration of the enzyme in the membrane when it is expressed as a portion of the membrane-bound NGFR/CDy construct. As the NGFR/CDy protein is associated with increased killing of transduced cells when compared to the expression of the

### Example 6

### Eradication of Genetically Engineered Cells in vivo

In order to determine whether genetically engineered CEM cells could be effectively eradicated in vivo, NOD/Scid mice were utilized. After being exposed to 150 rads of radiation on day 1, four groups of mice were injected with saline (control) or 5×10⁶ engineered cells on day 2. The remaining three groups included non-CD containing group (LNGFRSN), E. coli fusion construct containing group (LNGCDeSN) and the Saccharomyces fusion construct-containing group (LNGCDsSN). The genetically engineered cells were suspended in PBS at a 5×10⁶ cells/0.5 ml and injected via the tail vein. Commencing day 5 though day 19, the mice were injected with 5-FC (Sigma, cat no. F-7129, lot no. 110K4012) at 400 mg/kg using a stock solution of 12.5 mg/ml, prepared in normal saline. After two weeks of 5-FC treatment, the mice were followed to assess mortality within each group. A Kaplan Meyer analysis was performed to determine the effect of 5-FC treatment for each group. The experiment was continued for 100 days after initiation.

As expected, control mice injected with saline survived the duration of the experiment. No adverse effects of 5-FC injections were noted. Mice injected with CEM cells transduced with LNGFRSN do not have any means to clear the cells when exposed to 5-FC. This group serves as a second control against mice injected with CD containing fusion constructs. Survival was statistically different (p<0.02) between this group and the group injected with the Saccharomyces containing fusion construct showing the eradication of the CEM cells subsequent to treatment with 5-FC (FIG. 8). This survival benefit was not observed for the mice injected with CEM cells engineered with the E. coli derived CD fusion construct (p=0.178) even though evidence of cell cytotoxicity was seen in vitro LNGCDeSN CEM cells exposed to 5-FC. The superior function of the LNGCDsSN engineered cells was further demonstrated in CD enzymatic assays using HPLC.

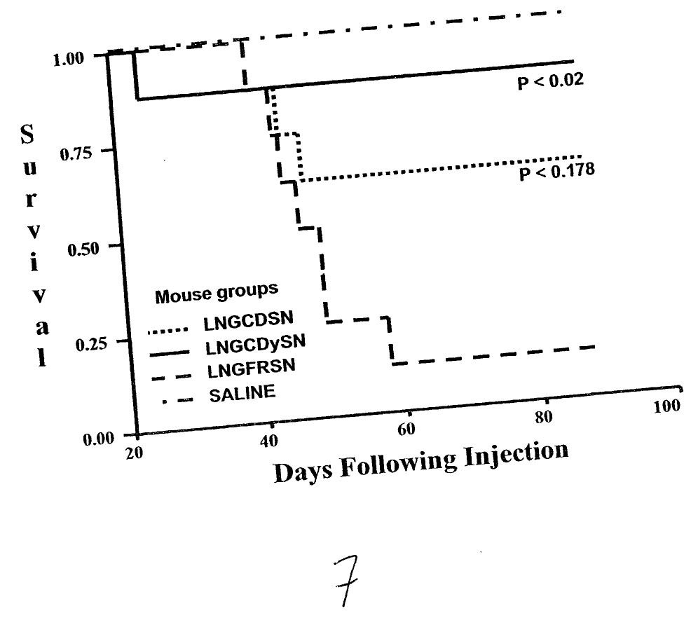

The remaining 3 groups consisted of mice receiving 5×10e⁶ CEM cells transduced with the LNGFRSN virus as a control, or an equal number of cells transduced with the LNGFR/CDeSN or LNGFR/CDsSN viruses. On day 5 through 19 the animals received an intraperitoneal injection of 400 mg/kg of 5-FC daily. In this experiment, control animals receiving injections of saline alone survived, while the vast majority of mice injected with CEM cells transduced with LNGFRSN died from disease progression. Survival was statistically different (p<0.02) between this group and the group injected with CEM cells expressing LNGFR/CDsSN with administration of 5-FC (FIG. 7). A statistically significant survival benefit was not observed for the mice injected with CEM cells transduced with the LNG/CDeSN vector (p=0.178). These studies confirm that the NGFR/CDs construct provides superior sensitivity to 5-FC in vivo as well as in vitro. The survival of mice receiving LNGFR/CDsSN modified CEM cells using a fourteen day treatment with 5-FC provides evidence that human T cells transduced with LNGFR/CDsSN can be eliminated in vivo. This confirms that the use of this construct may provide an alternative to the use of the HSV-tk construct for eliminating malignant cell populations in vivo, and suggests that it could be used as well as a means of controlling GvHD in a setting of allogeneic transplantation in which donor T cells are engineered to express this construct or in other gene therapy applications. This would allow the administration of acyclovir or ganciclovir in a clinical setting for the prophylaxis or treatment of viruses such as HSV or cytomegalovirus without eliminating engineered donor T cells.

### Example 7

### Eradication of bone cancers

Mice with subcutaneous or bone-residing cancers (2472 murine sarcoma) transduced with LNGFR/CDySN retrovirus were treated with 5-FC. Elimination of these cancers was observed in tumors transduced with the fusion construct containing S. cerevisiae CD (p<0.001). In addition to elimination of bone cancer, a complete blockage of tumor-induced osteolysis was also noted (p<0.0001).

When mixtures of non-transduced and LNGFR/CDySN retrovirus-transduced cancer cells were grown in vivo, only a fraction (10%) of transduced cells were required for therapeutic elimination of the tumor. This indicates that bystander killing of tumor cells occurs.

### Example 8

### Enhancement of 5-FC Sensitivity

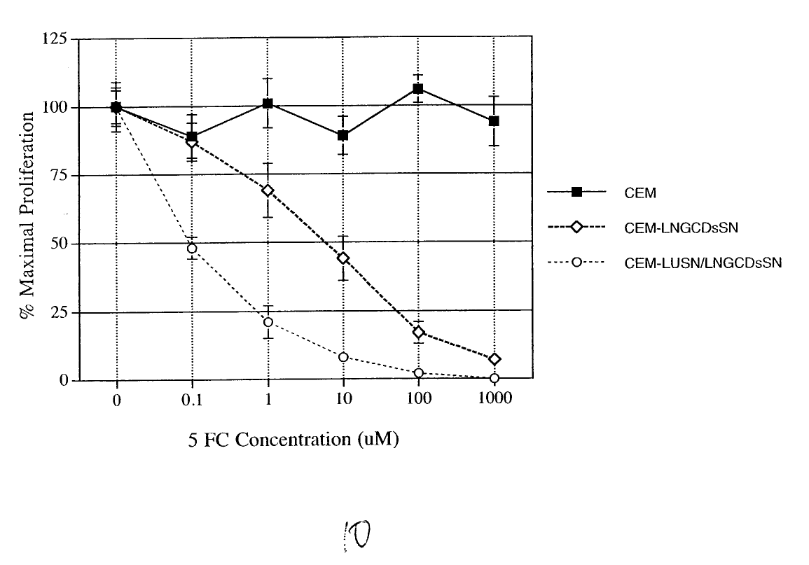

This Example describes the effects of expression of the UPRTase construct in combination with the NGFR/CDs construct in human T cells (CEM) on the sensitivity of the cells to 5-FC. Lines were transduced with a retrovirus containing the URPTase construct (LUSN). These cells were selected in neomycin. The LUSN-transduced cells were then transduced with the LNG/CDsSN retrovirus, and selected on the basis of the cell surface antigen NGFR. The doubly transduced cells were compared in this Example to cells transduced only with LNG/CDsSN, and the parental line. As depicted in FIG. 10, the doubly transduced cells are much more sensitive to 5-FC. This data demonstrates that a construct with all 3 elements (NGFR, CD and UPRT) will be very efficient in providing killing of transduced cells.

### Example 9

### Selection of Cells

Experiments comparing selection based on G418 and NGFR inactivated and transduced T-cells (data not shown) clearly demonstrate the superiority of the cell surface marker based technique. We have tested the Baxter Isolex 300i as well as the Miltenyi CliniMACS machine in experiments where transduced cells were divided between the two techniques. Whereas a better recovery was observed with the Baxter Isolex 300i system, the level of purity achieved using the CliniMACS magnetic beads has been found to be superior, though it failed to reach statistical significance, a trend in that direction was evident. We have thus opted to use the CliniMACS system to perform cell separation in a planned clinical trial using a different suicide construct that also has NGFR as a selection marker. In addition to superiority of selection of transduced cells, the NGFR molecule also has the advantage of likely being less immunogenic, as it has been documented that immunologic responses can be directed against antibiotic resistance genes (Riddell et al., 1996; Verzeletti et al., 1998).

### REFERENCES

ADACHI et al., Hum Gene Ther, 11, 77-89 (2000).

AGBARIA et al., Mol Pharmacol, 45, 777-782 (1994).

AMARA et al., Hum Gene Ther, 10, 2651-2655 (1999).

AUSTIN et al., Mol Pharmacol, 43, 380-387 (1993).

BONINI et al., Science, 276, 1719-1724 (1997).

COHEN et al., Blood, 89, 4636-4645 (1997).

CONSALVO et al., J Immunol, 154, 5302-5312 (1995).

CONTASSOT et al., Transplantation, 69, 503-508 (2000).

DANIELSEN et al., Mol Microbiol, 6, 1335-1344 (1992).

DAVIES et al., Leuk Lymphoma, 23, 221-226 (1996).

DAVISON et al., Transplantation, 69, 1341-1347 (2000).

DONG et al., Hum Gene Ther, 7, 713-720 (1996).

ELION, J Antimicrob Chemother, 12 Suppl B, 9-17 (1983).

ENRIGHT et al., Blood, 88, 714-720 (1996).

GALE et al., Bone Marrow Transplant, 6 Suppl 1, 94-97 (1990).

GALLARDO et al., Gene Ther, 4, 1115-1119 (1997).

GARIN et al., Blood, 97, 122-129 (2001).

HAMSTRA et al., Hum Gene Ther, 10, 1993-2003 (1999).

HIRSCHOWITZ et al., Hum Gene Ther, 6, 1055-1063 (1995).

HOGANSON et al., Cancer Res, 56, 1315-1323 (1996).

HOROWITZ et al., Blood, 75, 555-562 (1990).

HUBER et al., Cancer Res, 53, 4619-4626 (1993).

HUBER et al., Proc Natl Acad Sci USA, 91, 8302-8306 (1994).

LJUNGMAN et al., Bone Marrow Transplant, 12, 399-403 (1993).

MAR et al., J Virol, 56, 846-851 (1985).

MARMONT et al., Blood, 78, 2120-2130 (1991).

MARTIN et al., Blood, 66, 664-672 (1985).

MULLEN et al., Cancer Res, 54, 1503-1506 (1994).

NADEMANEE et al., Blood, 86, 1228-1234 (1995).

PAQUIN et al., Hum Gene Ther, 12, 13-23 (2001).

RICHARDS et al., Hum Gene Ther, 6, 881-893 (1995).

RIDDELL et al., Nat Med, 2, 216-223 (1996).

ROWLEY et al., J Surg Oncol, 61, 42-48 (1996).

THOMIS et al., Blood, 97, 1249-1257 (2001).

TIBERGHIEN et al., J Intern Med, 249, 369-377 (2001).

TIBERGHIEN et al., Hum Gene Ther, 8, 615-624 (1997).

TIBERGHIEN et al., Blood, 97, 63-72 (2001).

TRINH et al., Cancer Res, 55, 4808-4812 (1995).

VERZELETTI et al., Hum Gene Ther, 9, 2243-2251 (1998).

YAMAMOTO et al., Cancer Gene Ther, 4, 91-96 (1997).

ZITVOGEL et al., Hum Gene Ther, 5, 1493-1506 (1994).

All publications, patents and patent documents are incorporated by reference herein, as though individually incorporated by reference. The invention has been described with reference to various specific and preferred embodiments and techniques. However, it should be understood that many variations and modifications may be made while remaining within the scope of the invention.

What is claimed is:

1. A chimeric vector, comprising a first nucleic acid region encoding an extracellular domain of a protein, and a second nucleic acid region encoding a cytosine deaminase (CD), operably linked to the first region.

2. The vector of claim 1, wherein the first region further encodes a transmembrane domain of a protein.

3. The vector of claim 1, wherein the first region encodes a human or murine extracellular domain.

4. The vector of claim 2, wherein the first region encodes the transmembrane and extracellular domains of the human nerve growth factor receptor (NGFR).

5. The vector of claim 1, wherein the second region encodes a eukaryotic CD.

6. The vector of claim 5, wherein the second region encodes a yeast CD.

7. The vector of claim 6, wherein the second region encodes a Saccharomyces CD.

8. The vector of claim 7, wherein the second region encodes a Saccharomyces cerevisiae CD.

9. The vector of claim 1, wherein the CD is a humanized CD.

10. The vector of claim 1, further comprising a third nucleic acid region encoding a uracil phosphoribosyltransferase (UPRT).

11. The vector of claim 10, wherein the UPRT is a Toxoplasma gondi UPRT.

12. The vector of claim 10, wherein the UPRT is a Saccharomyces cerevisiae UPRT.

13. The vector of claim 1, further comprising another nucleic acid region encoding a linker region operably linked to the first and second regions.

14. The vector of claim 13, wherein the linker is a (gly₄ser)2 linker.

15. The vector of claim 1, further comprising another nucleic acid region encoding a sequence that, when expressed, imparts a therapeutic phenotype.

16. A host cell, comprising the vector of claim 1.

17. The host cell of claim 16, which is a T lymphocyte.

18. A transgenic non-human animal, comprising vector of claim 1.

19. A transgenic non-human animal, comprising cell of claim 16.

20. The transgenic animal of claim 19, which is a mouse.

21. A method of preventing or treating graft versus host disease (GVHD) in a patient, comprising:
(a) administering cells of claim 17 to the patient;
(b) determining or detecting the presence of the cells of claim 17 in a biological sample from the patient; and
(c) correlating the presence of the cells of claim 17 against any clinical symptoms of GVHD present in the patient

22. The method of claim 21, further comprising readministering cells of claim 17 to the patient.

23. The method of claim 21, wherein the cells are administered to the patient after the patient has received a bone-marrow transplant.

24. The method of claim 21, wherein the cells of claim 17 are determined or detected by fluorescence-activated cell sorting (FACS).

25. The method of claim 21, wherein the cells of claim 17 are determined or detected by magnetic immunobeads conjugated to antibodies.

26. The method of claim 21, further comprising administering 5-fluorocytosine (5-FC) to the patient in an amount effective to cause the elimination of the cells of claim 17.

27. The method of claim 21, wherein the cells of claim 17 are transduced lymphocytes from the patient.

28. A T lymphocyte comprising a first nucleic acid segment encoding the transmembrane and extracellular domains of the human nerve growth factor receptor, a second nucleic acid segment encoding a Saccharomyces cerevisiae cytosine deaminase, and third nucleic acid segment encoding a (gly₄ser)2 linker, operably linked to the first and second nucleic acid segments.

29. The lymphocyte of claim 28 further comprising a nucleic acid segment encoding a uracil phosphoribosyltransferase.

30. A nucleic acid sequence comprising a first nucleic acid segment encoding the transmembrane and extracellular domains of the human nerve growth factor receptor, a second nucleic acid segment encoding a Saccharomyces cerevisiae cytosine deaminase, and third nucleic acid segment encoding a (gly4ser)₂ linker, operably linked to the first and second nucleic acid segments.

31. The nucleic acid sequence of claim 30 further comprising a nucleic acid segment encoding a uracil phosphoribosyltransferase.

32. A polypeptide encoded by the nucleic acid sequence of claim 30 or 31.
